(19) United States
(12) Patent Application Publication
(10) Pub. No.: US 2006/0024703 A1
(43) Pub. Date: Feb. 2, 2006

(54) Library on a slide and the use thereof

(75) Inventors: Lixin Zhang, Ann Arbor, MI (US); Betsy Foxman, Ann Arbor, MI (US); Carl F. Marrs, Ann Arbor, MI (US); Janet R. Gilsdorf, Ann Arbor, MI (US); Usha Srinivasan, Ann Arbor, MI (US); Debashis Ghosh, Ann Arbor, MI (US)

Correspondence Address: David A. Casimir;MEDLEN & CARROLL, LLP, Suite 350, 101 Howard Street, San Francisco, CA, 94105, US

(73) Assignee: The Regents of the University of Michigan, Ann Arbor, MI (US)

(21) Appl. No.: 11/142,590

(22) Filed: Jun. 1, 2005

Related U.S. Application Data
(60) Provisional application No. 60/575,911, filed on Jun. 1, 2004.

(51) Int. Cl. C12Q 1/68; C12M 1/34; C40B 40/08

(52) U.S. Cl. 435/6; 435/287.2

(57) ABSTRACT

The present invention relates to compositions and methods for the detection and characterization of nucleic acid sequences and variations in nucleic acid sequences present in multiple genomes. In particular, the present invention provides microarrays possessing two or more whole genomes and methods of making and using the same to detect the presence or absence of target sequences in the plurality of genomes.

The present invention claims priority to U.S. Provisional Patent Application Ser. No. 60/575,911, filed Jun. 1, 2004, the disclosure of which is herein incorporated by reference in its entirety.

This invention was funded, in part, under NIH Grants AI054406, DK055496, AI51675 and DC005840. The government may have certain rights in the invention.

### FIELD OF THE INVENTION

The present invention relates to compositions and methods for the detection and characterization of nucleic acid sequences and variations in nucleic acid sequences present in multiple genomes. In particular, the present invention provides microarrays possessing two or more genomes and methods of making and using the same to detect the presence or absence of target sequences in the plurality of genomes.

### BACKGROUND OF THE INVENTION

Bacteria, viruses, and other pathogens produce a spectrum of genetic variants that contribute to diverse host specificity and pathogenicity. Genetic variants are marked not only by within-species variation in gene sequences, but more importantly, by their specific gene content. For example, even strains of the same species may differ by as much as 25% in genetic material (See e.g., Bergthorsson and Ochman, J Bacteriol, 177, 5784(1995); Bergthorsson and Ochman, Mol Biol Evol, 15, 6 (1998)). Horizontal transfer of genes from the same or related species, different gene alleles, transposon or phage related sequences, and extrachromosomal elements contribute to these differences. Each difference may be important for an organism's specific life style and pathogenic potential. The presence or absence of pathogenicity islands (See e.g., Lee, et al., Infect Agen Dis, 5,1 (1996); Hacker et al., Mol Microbiol, 23,1089 (1997)) on the genomes of pathogenic strains of bacteria is one example of gene content defining biological properties. Comparing gene frequencies among isolates collected from different sources (e.g., disease causing and commensal isolates) serves as a valuable strategy to gain insight into the relative importance of a gene sequence in pathogenesis, transmission and other biologically significant properties (See e.g., Zhang et al., Infect Immun, 68, 2009, (2000)). The populations studied, and the number of isolates are important in determining the significance of observations made and the power to detect associations. These comparisons are currently accomplished by membrane-based dot blot screening, a relatively low throughput, time consuming and laborious process.

The study of large numbers of strains is required to determine the relative frequency of various genes within a species and to gain insight into their association with pathogenesis, antibiotic resistance, adaptation to environmental factors, and transmission. Large population based samples are required to minimize the identification of spurious associations that often arise with small and convenient sample comparisons. Hence, researches need an affordable, robust and exacting way to efficiently examine large numbers of entire genomes (e.g., bacterial, viral, fungal, etc.) for the presence or absence of gene content defining biological properties.

### SUMMARY OF THE INVENTION

The present invention provides compositions and methods for the detection and characterization of nucleic acid sequences and variations in nucleic acid sequences present in multiple genomes. In particular, the present invention provides microarrays possessing two or more genomes and methods of making and using the same to detect the presence or absence of target sequences in the plurality of genomes.

Accordingly, in some embodiments, the present invention provides a composition comprising two or more genomes affixed to a solid surface. In other embodiments, the present invention provides a composition comprising a plurality of whole genomes provided as a microarray on a solid surface. In some embodiments, the composition of two or more genomes comprise total genomic nucleic acid. In other embodiments, the two or more genomes comprise total genomic DNA or total genomic RNA. In some embodiments, the total nucleic acid, total genomic DNA or total genomic RNA comprises total nucleic acid, DNA, or RNA, derived from multiple subjects, strains, isolates, or species. In some embodiments, the total nucleic acid, total genomic DNA or total genomic RNA comprises total nucleic acid, DNA, or RNA, derived from a single subject, strain, isolate, or specie. In some embodiments, the subject, strain, isolate or specie is selected from the group comprising humans, bacteria, viruses, yeast, algae, fungi, animals and plants. In some embodiments, the two or more genomes are fragmented. In some embodiments, the fragmented genomes are substantially composed of fragments 0.1 kb-10 kb in length. In preferred embodiments, the fragmented genomes are substantially composed of fragments 0.05 kb-1.0 kb in length. In other embodiments, the fragments are 1.0 kb-10 kb in length. In still other embodiments, the fragments are 2.0 kb-10 kb in length. In a preferred embodiment, the fragments are 2.0 kb-5.0 kb in length.

In a preferred embodiment, the solid surface to which the two or more genomes are affixed is glass. The present invention is not limited by the type of solid surface chosen. Indeed, a variety of solid surfaces are useful in the present invention, including, but not limited to, silicon, plastic, polymer, ceramic, photoresist, nitrocellulose, hydrogel, paper, polypropylene, polystyrene, nylon, polyacrylamide, optical fiber, natural fibers, nylon, metals, rubber and composites thereof. In some embodiments, the solid surface comprises more than one type of solid surface. For example, in some embodiments the solid surface comprises both glass and nylon (e.g., modified nylon polymers), or any other combination of materials useful for making a surface suitable for application of genomic arrays. In a preferred embodiment, the two or more genomes are spotted in arrays on the solid surface. In some embodiments, the solid surface size is 20 mm×60 mm or smaller, although the present invention is not limited by the size of the solid surface (both larger and smaller surfaces are are useful, in one or more dimensions). In some embodiments, there are at least 10 genomes spotted in arrays on the solid surface. The present invention provides the spotting of large numbers of genomes onto the solid surface. In some embodiments, at least 100 genomes are spotted in arrays on the solid surface. In other embodiments, at least 1,000 genomes are spotted in arrays on the solid surface. In some embodiments, at least 3,000 genomes are spotted in arrays on the solid surface. In other embodiments, at least 10,000 genomes are spotted in arrays on the solid surface. In still further embodiments, at least 30,000 genomes are spotted in arrays on the solid surface.

In some embodiments, the solid surface is planer. In a preferred embodiment, the solid surface is glass. In a particularly preferred embodiment, the glass is a glass slide. The present invention is not limited to a particular type of solid surface. Indeed a variety of solid surfaces find use in the present invention, including a solid surface that comprises a plurality of microfluidic channels. In some embodiments, the microfluidic channels are one-dimensional line arrays. In other embodiments, the microfluidic channels are two-dimensional arrays. In still other embodiments, the solid surface further comprises a plurality of etched microchannels or pores or wells. In some embodiments, the solid surface is in a two-dimensional configuration or a three-dimensional configuration comprising pins, rods, fibers, tapes, threads, sheets, films, gels, membranes, beads, plates, particles, microtiter wells, capillaries, or cylinders.

In another embodiment, the present invention provides a nucleic acid array, the nucleic acid array comprising a solid support and a plurality of whole genomes, each of the whole genomes affixed to the solid support at a predetermined location, and each of the whole genomes comprising total genomic DNA and/or RNA, the total genomic DNA and/or RNA derived from a single individual, strain, isolate or species of humans, bacteria, viruses, yeast, algae, fungi, animals or plants, wherein the total genomic DNA or RNA is fragmented.

The present invention also provides a method for detecting a target sequence in a plurality of genomes comprising providing a composition comprising two or more genomes affixed to a solid surface; a probe specific for a target sequence; and hybridizing the probe to the composition under conditions such that the presence or absence of the sequence in the two or more genomes is identified. In some embodiments, the target sequence in the plurality of genomes comprises nucleic acid sequence. In a preferred embodiment, the genomes comprise genomes from pathogens. In other preferred embodiments, the target sequence is a gene associated with antibiotic susceptibility or resistance. In some embodiments, the target sequence is a transposable element. In still other embodiments, the target sequence encodes all or part of a nucleic acid sequence of interest, including, but not limited to, sequences of virulence genes, antibiotic resistant genes, transposable elements, genes with single nucleotide mutations, genes with single nucleotide polymorphisms, genes with deletions, genes with insertions, and genes with mutations.

In a preferred embodiment, the probe specific for a target sequence is single stranded DNA. The present invention is not limited by the nature of the probe used. Indeed a variety of probes find use in the present invention including oligonucleotide, DNA, amplified DNA, cDNA, double stranded DNA, PNA, RNA, and mRNA probes. In some embodiments, the probe is less than 100 bp. In other embodiments, the probe is 0.1 kb-1.0 kb. In still other embodiments, the probe is 1.0 kb-5.0 kb. In other embodiments, the probe is 5.0 kb-7.0 kb. In some embodiments, the probe is 7.0 kb-10 kb. In some embodiments, the probe is greater than 10 kb. In a preferred embodiment, the probe contains a capture sequence (e.g., a dendrimer capture sequence). In other preferred embodiments, the probe is detectably labeled with fluorescent dyes or other labels. In particularly preferred embodiments, the fluorescent dyes include, but are not limited to, fluorescein dyes, rhodamine dyes, BODIPY, and Cy3 or Cy5 dyes. The present invention is not limited to a particular type of label. Indeed, a variety of detectable labels find use in the present invention including, but not limited to, biotin, magnetic beads, radiolabels, enzymes, colorimetric labels and plastic beads.

In some embodiments, the identification of the presence or absence of the target sequence in the plurality of genomes is standardized using a dual channel non-competing hybridization strategy. In further embodiments, the dual channel non-competing hybridization strategy utilizes signals generated by 16s rRNA.

The present invention also provides a method for detecting a sequence in a genome, comprising providing a composition comprising a plurality of whole genomes provided as a microarray on a solid surface and a probe specific for a target sequence; and hybridizing the probe to the composition under conditions such that the presence or absence of the target sequence in the genome is identified. The present invention also provides a method of comparing genomes for the presence or absence of one or more sequences, the method comprising contacting a microarray comprising a plurality of whole genomes derived from different sources with one or more nucleic acid probes and identifying the genome or genomes to which the probe(s) binds. In some embodiments, the microarray comprises two or more genomes derived from a single type of bacteria, virus, fungus, yeast or algae, but under different forms of environmental stress. In further embodiments, the environmental stress comprises heat shock, low temperature, amino acid depletion, ultraviolet radiation or exposure to antibiotics.

The invention also provides a kit comprising a composition comprising a plurality of whole genomes provided as a microarray on a solid surface. In some embodiments, the kit comprises instructions for using the microarray, wherein the instructions are for determining the presence or absence of a target sequence within one or more of the plurality of whole genomes. In other embodiments, the kit comprises probes specific for binding to a target sequence within one or more of the plurality of whole genomes. In further embodiments, the probe is selected from a group consisting of an oligonucleotide, DNA, amplified DNA, cDNA, single stranded DNA, double stranded DNA, PNA, RNA, and mRNA.

The present invention also provides a method of making an array wherein two or more genomes are affixed to a solid surface. In some embodiments, the two or more genomes comprise total genomic nucleic acid. In other embodiments, the two or more genomes comprise total genomic DNA or total genomic RNA, the total genomic DNA or total genomic RNA derived from a single individual, strain, isolate or species of humans, bacteria, viruses, yeast, algae, fungi, animals or plants. In some embodiments, the solid surface is selected from the group consisting of silicon, plastic, polymer, ceramic, photoresist, nitrocellulose, hydrogel, paper, polypropylene, polystyrene, nylon, polyacrylamide, optical fiber, natural fibers, nylon, metals, rubber and composites thereof. In a preferred embodiment, the solid surface is glass. In some embodiments, the solid surface comprises a plurality of etched microchannels. In other embodiments, the solid surface is in a two-dimensional configuration or a three-dimensional configuration comprising pins, rods, fibers, tapes, threads, sheets, films, gels, membranes, beads, plates, particles, microtiter wells, capillaries, or cylinders. In some embodiments, the total genomic DNA or total genomic RNA is highly purified. In some embodiments, the purification comprises organic extraction. In some embodiments, the purification comprises the use of membranes and resins. In a preferred embodiment, the two or more genomes are fragmented. In some embodiments, the fragmented genomes are substantially composed of fragments 0.1 kb-10 kb in length. In preferred embodiments, the fragmented genomes are substantially composed of fragments 0.05 kb-1.0 kb in length. In other embodiments, the fragments are 1.0 kb-10 kb in length. In still other embodiments, the fragments are 2.0 kb-10 kb in length. In a preferred embodiment, the fragments are 2.0 kb-5.0 kb in length. In another preferred embodiment, the fragmented two or more genomes are spotted onto a solid surface. In some embodiments, the solid surface size is 20 mm×60 mm or smaller. In some embodiments, there are at least 10 genomes spotted in arrays on the solid surface. The present invention provides the spotting of large numbers of genomes onto the solid surface. In some embodiments, at least 100 genomes are spotted in arrays on the solid surface. In other embodiments, at least 1,000 genomes are spotted in arrays on the solid surface. In some embodiments, at least 3,000 genomes are spotted in arrays on the solid surface. In other embodiments, at least 10,000 genomes are spotted in arrays on the solid surface. In still further embodiments, at least 30,000 genomes are spotted in arrays on the solid surface. The present invention further provides a composition created by the method of making an array comprising two or more genomes affixed to a solid surface.

### DESCRIPTION OF THE FIGURES

FIGS. 1A-B show the signal intensities of a two fold genomic DNA dilution series probed with (A) a 1 kb or (B) a 7 kb direct labeled hly Cy5 probe. The darker dots represent spotting concentrations from 4 μg/ul to 0.125 μg/ul plus a negative control (the last spot in the series). The lighter line represents the simulated ideal signal responding line for a 2 fold dilution series that covers the whole signal spectrum of the scanner (16 bit image). The last dark spot in the series represents the background signal.

FIGS. 2A-C show a test array of the E. coli J96 genomic DNA hybridized with (A) a Cy3 direct labeled 1 kb hly gene probe prepared with random priming (very light signal detected higher concentration spots), (B) a single stranded 1 kb hly gene fragment with a 5′ capture sequence and detected by Cy3 DNA Dendrimer, or (C) a fluorescein labeled 1 kb hly probe and detected with Tyramide Signal Amplification (TSA) system.

FIGS. 3A-D show an E. coli reference collection (ECOR) library array simultaneously probed with (A) a green fluorescence labeled hly probe and (B) a red fluorescence labeled quantification probe, the 16s rRNA gene. Four sub-grids of the 2352 spots shown in each (A) and (B) are shown in (C) and (D),respectively, each with 98 spots.

FIG. 4 shows scatter plots of the average percentage signal intensities adjusted according to the 16 sRNA probe (TOP) and unadjusted signal values compared to the positive control (BOTTOM).

FIG. 5A shows (1) a cell suspension after sonication, (2) a suspension pelleted down by centrifugation, and (3) a precipitation out of supernant from 2 after heat treatment. FIG. 5B shows gel electrophoresis of DNA obtained from 6 bacterial strains (lanes 1,2—E. coli; lanes 3,4—H. influenzae; lanes 5,6—S. agalactiae) using the sonication based method of the present invention. FIG. 5C, panel 1 shows a glass array printed with genomic DNA from 15 E. coli isolates probed with Cy3 labeled 16sRNA gene probe. FIG. 5C, panel 2 shows a glass array printed with 8 PCR amplified ORFs (from left to right and top to bottom: hlyA, hlyB, draA fimH, papG, papI, papa, fimA; only draA is absent in this genome) probed with Cy3 labeled CFT073 genomic DNA. FIG. 5D shows PCR amplification of DNA fragments of various sizes (lanes 1,2—390 bp fimA; lanes 3,4—1043 bp hlyA; lanes 5,6—1.4 kb rrsA) using CFT073 genomic DNA isolated using the sonication method of the present invention.

### DEFINITIONS

As used herein, the term “spotting” or “tapping,” with respect to depositing a genome on a microarray surface, refers to contacting the surface with a device, such as a microarray printing pin, containing a genome such that the genome is deposited on the surface and is in contact with the surface of the microarray at a defined, preferably discrete position. Preferably, the spotting or tapping is via a capillary or other tube (such as within the printing pin) capable of depositing a small volume of solution comprising genomes on the surface, wherein the volume is 1 μl or less, 100 nl or less, 10 nl or less, 5 nl or less, 2 nl or less, 1 nl or less, or 0.5 nl or less. Preferably the spot formed by depositing the genome solution on the surface is separated from other spots on the microarray such that subsequent hybridization or other reaction on the array is not adversely affected by reactions on neighboring or nearby spots. Preferably, the spot is from 50-500 microns, from 75-300 microns, or from 100-150 microns in diameter.

As used herein, the term “solid surface” refers to any solid surface suitable for the attachment of biological molecules and the performance of molecular interaction assays. Surfaces may be made of any suitable material (e.g., including, but not limited to, silicon, plastic, glass, polymer, ceramic, photoresist, nitrocellulose, hydrogel, paper, polypropylene, polystyrene, nylon, polyacrylamide, optical fiber, natural fibers, nylon, metals, rubber and composites or polymers thereof) and may be modified with coatings (e.g., metals or polymers). Furthermore, a solid surface may comprise two or more materials (e.g., glass and nylon). Solid surfaces need not be flat. Solid surfaces may include any three dimensional shape including pins, rods, fibers, tapes, threads, sheets, films, gels, membranes, beads, plates, particles, microtiter wells, capillaries, or cylinders. Materials attached to solid surfaces may be attached to any portion of the solid surface (e.g., may be attached to an interior portion of a porous solid support material). Additionally, the solid surface (e.g., glass) may be treated (e.g., amine or epoxy treated) for use in the present invention. Preferred embodiments of the present invention have biological molecules such as nucleic acid molecules attached to solid surfaces. The term “attached,” when used to describe a state of interaction between a biological material and a solid surface, describe non-random interactions including, but not limited to, covalent bonding, ionic bonding, chemisorption, physisorption and combinations thereof.

As used herein, the term “microarray” refers to a solid surface comprising a plurality of addressed biological macromolecules (e.g. nucleic acid sequences). Microarrays, are described generally, for example, in Schena, “Microarray Biochip Technology,” Eaton Publishing, Natick, Mass., 2000.

As used herein, the term “microfluidic channels” or “etched microchannels” refers to three-dimensional channels created in material deposited on a solid surface.

As used herein, the term “one-dimensional line array” refers to parallel microfluidic channels on top of a surface that are oriented in only one dimension.

As used herein, the term “two dimensional arrays” refers to microfluidic channels on top of a surface that are oriented in two dimensions. In some embodiments, channels are oriented in two dimensions that are perpendicular to each other.

As used herein, the term “microchannels” refers to channels etched into a surface. Microchannels may be one-dimensional or two-dimensional.

As used herein, the term “target sequence” refers to a nucleic acid molecule to be detected or characterized. In some embodiments, target nucleic acids contain a sequence that has at least partial complementarity with at least a probe oligonucleotide. The target nucleic acid may comprise single- or double-stranded DNA or RNA. Examples of target sequences include, but are not limited to, sequences of virulence genes, antibiotic resistant genes, transposable elements, genes with single nucleotide mutations, genes with single nucleotide polymorphisms, genes with deletions, genes with insertions, and genes with mutations.

The term “signal” as used herein refers to any detectable effect, such as would be caused or provided by an assay reaction. For example, in some embodiments of the present invention, signals are from labels such as fluorescent signals.

As used herein, the terms “SNP,” “SNPs” or “single nucleotide polymorphisms” refer to single base changes at a specific location in an organism's (e.g., a microorganism or a human) genome. “SNPs” can be located in a portion of a genome that does not code for a gene. Alternatively, a “SNP” may be located in the coding region of a gene. In this case, the “SNP” may alter the structure and function of the RNA or the protein with which it is associated.

As used herein, the term “allele” refers to a variant form of a given sequence (e.g., including but not limited to, genes containing one or more SNPs). A large number of genes are present in multiple allelic forms in a population. A diploid organism carrying two different alleles of a gene is said to be heterozygous for that gene, whereas a homozygote carries two copies of the same allele.

As used herein, the term “linkage” refers to the proximity of two or more markers (e.g., genes) on a chromosome.

As used herein, the term “allele frequency” refers to the frequency of occurrence of a given allele (e.g., a sequence containing a SNP) in a given population (e.g., of organisms, strains or species). Certain populations may contain a given allele within a higher percent of its members than other populations.

As used herein, the term “in silico analysis” refers to analysis performed using computer processors and computer memory. For example, “insilico SNP analysis” refers to the analysis of SNP data using computer processors and memory.

As used herein, the term “genotype” refers to the actual genetic make-up of an organism (e.g., in terms of the particular alleles carried at a genetic locus). Expression of the genotype gives rise to an organism's physical appearance and characteristics—the “phenotype.”

As used herein, the term “locus” refers to the position of a gene or any other characterized sequence on a chromosome.

The term “gene” refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in, corresponding portions of the produced RNAs (e.g., hnRNAs, mRNAs, rRNAs, tRNAs).

Where the phrase “amino acid sequence” is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

DNA and RNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

When used in reference to a single-stranded nucleic acid sequence, the term “substantially homologous” refers to any probe that can hybridize (i.e., it is the complement of) to the single-stranded nucleic acid sequence under conditions of low stringency as described above.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41 (% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5.times.SSPE (43.8 g/l NaCl, 6.9 g/l NaH₂PO₄H₂O and 1.85 g/l EDTA, pH adjusted to 7.4 with NaOH), 0.5% SDS, 5.times.Denhardt's reagent and 100 .mu.g/ml denatured salmon sperm DNA followed by washing in a solution comprising 0.1.times.SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5.times.SSPE (43.8 g/l NaCl, 6.9 g/l NaH₂PO₄H₂O and 1.85 g/l EDTA, pH adjusted to 7.4 with NaOH), 0.5% SDS, 5.times.Denhardt's reagent and 100 .mu.g/ml denatured salmon sperm DNA followed by washing in a solution comprising 1.0.times.SSPE, 1.0% SDS at 42° C. when a probe of about 500 nucleotides in length is employed.

“Low stringency conditions” comprise conditions equivalent to binding or hybridization at 42° C. in a solution consisting of 5.times.SSPE (43.8 g/l NaCl, 6.9 g/l NaH₂PO₄H₂O and 1.85 g/l EDTA, pH adjusted to 7.4 with NaOH), 0.1% SDS, 5.times. Denhardt's reagent (50.times. Denhardt's contains per 500 ml: 5 g Ficoll (Type 400, Pharamcia), 5 g BSA (Fraction V; Sigma)) and 100 .mu.g/ml denatured salmon sperm DNA followed by washing in a solution comprising 5.times. SSPE, 0.1% SDS at 42° C. when a probe of about 500 nucleotides in length is employed.

One skilled in the relevant understands that stringency conditions may be altered for probes of other sizes (See e.g., Anderson and Young, Quantitative Filter Hybridization, in Nucleic Acid Hybridization (1985) and Sambrook et al., Molecular Cloning: A Laboratory Manual, Cold Spring Harbor Press, NY (1989)).

As used herein, the term “probe” refers to an polynucleotide (i.e., a sequence of nucleotides), whether occurring naturally as in a purified restriction digest or produced synthetically, recombinantly or by PCR amplification, that is capable of hybridizing to another oligonucleotide of interest. A probe may be an oligonucleotide, DNA, amplified DNA, cDNA, single stranded DNA, double stranded DNA, PNA, RNA, or mRNA. Probes are useful in the detection, identification and isolation of particular nucleic acid sequences.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxgenin; luminogenic, phosphorescent or fluorogenic moieties; magnetic beads; enzymes; colorimetric labels; plastic beads; and fluorescent dyes (e.g., fluorescein dyes, rhodamine dyes, BODIPY, and Cy3 or Cy5) alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

As used herein, the term “detector” refers to a system or component of a system, e.g., an instrument (e.g. a camera, fluorimeter, charge-coupled device, scintillation counter, etc.) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection systems known in the art, or combinations thereof.

As used herein, the term “sample” is used in its broadest sense. In one sense, it is meant to include cells (e.g., human, bacterial, yeast, and fungi), an organism, a specimen or culture obtained from any source, as well as biological and environmental samples. Biological samples may be obtained from animals (including humans) and refers to a biological material or compositions found therein, including, but not limited to, bone marrow, blood, serum, platelet, plasma, interstitial fluid, urine, cerebrospinal fluid, nucleic acid, DNA, tissue, and purified or filtered forms thereof. Environmental samples include environmental material such as surface matter, soil, water, crystals and industrial samples. Such examples are not however to be construed as limiting the sample types applicable to the present invention.

As used herein, the term “organism” refers to any entity from which total genomic DNA and/or RNA can be derived. For example, organisms may be subjects, strains, isolates, or species. In some embodiments, a subject, strain, isolate or species may be selected from humans, bacteria, viruses, yeast, algae, fungi, animals and plants.

The terms “whole genome,” “genome,” “total genomic nucleic acid,” and the like refer to at least 80%, preferably 90%, more preferably approximately 100% of the total set of genes and nucleic acid sequences surrounding these genes carried by an organism, a cell or an organelle. The terms “whole genome,” “genome,” “total genomic nucleic acid,” can refer to genomic DNA and/or genomic RNA. Similarly, the terms “total genomic DNA” and “total genomic RNA” refer to at least 80%, preferably 90%, more preferably approximately 100% of the total DNA or RNA, respectively, carried by an organism, a cell or an organelle. It is understood that small portions of genomic nucleic acid may be lost during isolation or preparation, but that the remaining material, which constitutes substantially all of the genome is considered a “whole genome,” “genome,” or “total genomic nucleic acid.”

As used herein, the term “derived from different organisms,” such as samples or nucleic acids derived from different organisms refers to samples derived from multiple different organisms. For example, a blood sample comprising genomic DNA from a first person and a blood sample comprising genomic DNA from a second person are considered blood samples and genomic DNA samples that are derived from different organisms. In some embodiments, a sample comprising five genomes derived from different organisms is a sample that includes at least five samples from five different organisms. However, a sample may contain multiple samples from a given organism. For example, in some embodiments, a composition of the present invention (e.g., a microarray) may comprise two or more genomes derived from a single organism. In such cases, for example, total nucleic acid may be obtained from an organism at two or more different time points (e.g., before and after exposure to certain environmental stresses, or every 5 minutes for 24 hours).

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements include splicing signals, polyadenylation signals, termination signals, etc.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polynucleotides, the term “substantial identity” denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a splice variant of the full-length sequences.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al, Molecular Cloning: A Laboratory Manual, Cold Spring Harbor Press, NY, pp 9.31-9.58 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of labeled antibodies.

The term “test compound” refers to any chemical entity, pharmaceutical, drug, and the like that are tested in an assay (e.g., a drug screening assay) for any desired activity (e.g., including but not limited to, the ability to treat or prevent a disease, illness, sickness, or disorder of bodily function, or otherwise alter the physiological or cellular status of a sample). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acids encoding a polypeptide include, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 11, . . . , 20, . . . ).

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “signal” as used herein refers to any detectable effect, such as would be caused or provided by a label or an assay reaction.

As used herein, the term “container” is used in its broadest sense, and includes any material useful for holding a sample or organism. A container need not be completely enclosed. Containers include tubes (e.g., eppendorf or conical tubes), plates, wells, microtiter plate wells, or any material capable of separating one sample from another (e.g., a microfluidic channel or engraved space on a solid surface). Such examples are not however to be construed as limiting the containers applicable to the present invention.

As used herein, the term “detector” refers to a system or component of a system, e.g., an instrument (e.g. a camera, fluorimeter, charge-coupled device, scintillation counter, etc) or a reactive medium (X-ray or camera film, pH indicator, etc.), that can convey to a user or to another component of a system (e.g., a computer or controller) the presence of a signal or effect. A detector can be a photometric or spectrophotometric system, which can detect ultraviolet, visible or infrared light, including fluorescence or chemiluminescence; a radiation detection system; a spectroscopic system such as nuclear magnetic resonance spectroscopy, mass spectrometry or surface enhanced Raman spectrometry; a system such as gel or capillary electrophoresis or gel exclusion chromatography; or other detection system known in the art, or combinations thereof.

The term “detection” as used herein refers to quantitatively or qualitatively identifying an analyte (e.g., DNA, RNA) within a sample. The term “detection assay” as used herein refers to a kit, test, or procedure performed for the purpose of detecting a nucleic acid within a sample. Detection assays produce a detectable signal or effect when performed in the presence of the target nucleic acid, and include but are not limited to assays incorporating the processes of hybridization, nucleic acid cleavage (e.g., exo- or endonuclease), nucleic acid amplification, nucleotide sequencing, primer extension, or nucleic acid ligation.

As used herein, the term “functional detection oligonucleotide” refers to an oligonucleotide that is used as a component of a detection assay, wherein the detection assay is capable of successfully detecting (i.e., producing a detectable signal) an intended target nucleic acid when the functional detection oligonucleotide provides the oligonucleotide component of the detection assay. This is in contrast to a non-functional detection oligonucleotides, which fail to produce a detectable signal in a detection assay for the particular target nucleic acid when the non-functional detection oligonucleotide is provided as the oligonucleotide component of the detection assay. Determining if an oligonucleotide is a functional oligonucleotide can be carried out experimentally by testing the oligonucleotide in the presence of the particular target nucleic acid using the detection assay.

As used herein, the term “treating together”, when used in reference to experiments or assays, refers to conducting experiments concurrently or sequentially, wherein the results of the experiments are produced, collected, or analyzed together (i.e., during the same time period). For example, a plurality of different genomes located in different portions of a microarray are treated together in a detection assay where detection reactions are carried out on the genomes simultaneously or sequentially and where the data collected from the assays is analyzed together.

The terms “assay data” and “test result data” as used herein refer to data collected from performance of an assay (e.g., to detect or quantitate a gene, SNP or an RNA). Test result data may be in any form, i.e., it may be raw assay data or analyzed assay data (e.g., previously analyzed by a different process). Collected data that has not been further processed or analyzed is referred to herein as “raw” assay data (e.g., a number corresponding to a measurement of signal, such as a fluorescence signal from a spot on a chip or a reaction vessel, or a number corresponding to measurement of a peak, such as peak height or area, as from, for example, a mass spectrometer, HPLC or capillary separation device), while assay data that has been processed through a further step or analysis (e.g., normalized, compared, or otherwise processed by a calculation) is referred to as “analyzed assay data” or “output assay data”.

As used herein, the term “database” refers to collections of information (e.g., data) arranged for ease of retrieval, for example, stored in a computer memory. A “genomic information database” is a database comprising genomic information, including, but not limited to, polymorphism information (i.e., information pertaining to genetic polymorphisms), genome information (i.e., genomic information), linkage information (i.e., information pertaining to the physical location of a nucleic acid sequence with respect to another nucleic acid sequence, e.g., in a chromosome), pathogenicity information (i.e., information related to nucleic acid sequence and ability to cause disease), and disease association information (i.e., information correlating the presence of or susceptibility to a disease to a physical trait of a subject, e.g., an allele of a subject). “Database information” refers to information to be sent to a databases, stored in a database, processed in a database, or retrieved from a database. “Sequence database information” refers to database information pertaining to nucleic acid sequences. As used herein, the term “distinct sequence databases” refers to two or more databases that contain different information than one another. For example, the dbSNP and GenBank databases are distinct sequence databases because each contains information not found in the other.

As used herein the terms “processor” and “central processing unit” or “CPU” are used interchangeably and refer to a device that is able to read a program from a computer memory (e.g., ROM or other computer memory) and perform a set of steps according to the program.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “hypertext system” refers to a computer-based informational system in which documents (and possibly other types of data entities) are linked together via hyperlinks to form a user-navigable “web.”

As used herein, the term “Internet” refers to any collection of networks using standard protocols. For example, the term includes a collection of interconnected (public and/or private) networks that are linked together by a set of standard protocols (such as TCP/IP, HTTP, and FTP) to form a global, distributed network. While this term is intended to refer to what is now commonly known as the Internet, it is also intended to encompass variations that may be made in the future, including changes and additions to existing standard protocols or integration with other media (e.g., television, radio, etc). The term is also intended to encompass non-public networks such as private (e.g., corporate) Intranets.

As used herein, the terms “World Wide Web” or “web” refer generally to both (i) a distributed collection of interlinked, user-viewable hypertext documents (commonly referred to as Web documents or Web pages) that are accessible via the Internet, and (ii) the client and server software components which provide user access to such documents using standardized Internet protocols. Currently, the primary standard protocol for allowing applications to locate and acquire Web documents is HTTP, and the Web pages are encoded using HTML. However, the terms “Web” and “World Wide Web” are intended to encompass future markup languages and transport protocols that may be used in place of (or in addition to) HTML and HTTP.

As used herein, the term “web site” refers to a computer system that serves informational content over a network using the standard protocols of the World Wide Web. Typically, a Web site corresponds to a particular Internet domain name and includes the content associated with a particular organization. As used herein, the term is generally intended to encompass both (i) the hardware/software server components that serve the informational content over the network, and (ii) the “back end” hardware/software components, including any non-standard or specialized components, that interact with the server components to perform services for Web site users.

As used herein, the term “HTML” refers to HyperText Markup Language that is a standard coding convention and set of codes for attaching presentation and linking attributes to informational content within documents. HTML is based on SGML, the Standard Generalized Markup Language. During a document authoring stage, the HTML codes (referred to as “tags”) are embedded within the informational content of the document. When the Web document (or HTML document) is subsequently transferred from a Web server to a browser, the codes are interpreted by the browser and used to parse and display the document. Additionally, in specifying how the Web browser is to display the document, HTML tags can be used to create links to other Web documents (commonly referred to as “hyperlinks”).

As used herein, the term “XML” refers to Extensible Markup Language, an application profile that, like HTML, is based on SGML. XML differs from HTML in that: information providers can define new tag and attribute names at will; document structures can be nested to any level of complexity; any XML document can contain an optional description of its grammar for use by applications that need to perform structural validation. XML documents are made up of storage units called entities, which contain either parsed or unparsed data. Parsed data is made up of characters, some of which form character data, and some of which form markup. Markup encodes a description of the document's storage layout and logical structure. XML provides a mechanism to impose constraints on the storage layout and logical structure, to define constraints on the logical structure and to support the use of predefined storage units. A software module called an XML processor is used to read XML documents and provide access to their content and structure.

As used herein, the term “HTTP” refers to HyperText Transport Protocol that is the standard World Wide Web client-server protocol used for the exchange of information (such as HTML documents, and client requests for such documents) between a browser and a Web server. HTTP includes a number of different types of messages that can be sent from the client to the server to request different types of server actions. For example, a “GET” message, which has the format GET, causes the server to return the document or file located at the specified URL.

As used herein, the term “URL” refers to Uniform Resource Locator that is a unique address that fully specifies the location of a file or other resource on the Internet. The general format of a URL is protocol://machine address:port/path/filename. The port specification is optional, and if none is entered by the user, the browser defaults to the standard port for whatever service is specified as the protocol. For example, if HTTP is specified as the protocol, the browser will use the HTTP default port of 80.

As used herein, the term “PUSH technology” refers to an information dissemination technology used to send data to users over a network. In contrast to the World Wide Web (a “pull” technology), in which the client browser must request a Web page before it is sent, PUSH protocols send the informational content to the user computer automatically, typically based on information pre-specified by the user.

As used herein, the term “communication network” refers to any network that allows information to be transmitted from one location to another. For example, a communication network for the transfer of information from one computer to another includes any public or private network that transfers information using electrical, optical, satellite transmission, and the like. Two or more devices that are part of a communication network such that they can directly or indirectly transmit information from one to the other are considered to be “in electronic communication” with one another. A computer network containing multiple computers may have a central computer (“central node”) that processes information to one or more sub-computers that carry out specific tasks (“sub-nodes”). Some networks comprises computers that are in “different geographic locations” from one another, meaning that the computers are located in different physical locations (i.e., aren't physically the same computer, e.g., are located in different countries, states, cities, rooms, etc.).

As used herein, the term “detection assay component” refers to a component of a system capable of performing a detection assay. Detection assay components include, but are not limited to, hybridization probes, buffers, and the like.

As used herein, the term “a detection assays configured for target detection” refers to a collection of assay components that are capable of producing a detectable signal when carried out using the target nucleic acid. For example, a detection assay that has empirically been demonstrated to detect a particular single nucleotide polymorphism is considered a detection assay configured for target detection.

As used herein, the phrase “unique detection assay” refers to a detection assay that has a different collection of detection assay components in relation to other detection assays located on the same detection panel. A unique assay doesn't necessarily detect a different target (e.g. SNP) than other assays on the same detection panel, but it does have a least one difference in the collection of components used to detect a given target (e.g. a unique detection assay may employ a probe sequences that is shorter or longer in length than other assays on the same detection panel).

As used herein, the term “candidate” refers to an assay or analyte, e.g., a nucleic acid, suspected of having a particular feature or property. A “candidate sequence” refers to a nucleic acid suspected of comprising a particular sequence, while a “candidate oligonucleotide” refers to an oligonucleotide suspected of having a property such as comprising a particular sequence, or having the capability to hybridize to a target nucleic acid or to perform in a detection assay. A “candidate detection assay” refers to a detection assay that is suspected of being a valid detection assay.

As used herein, the term “detection panel” refers to a substrate or device containing at least two unique candidate detection assays configured for target detection.

As used herein, the term “valid detection assay” refers to a detection assay that has been shown to accurately predict an association between the detection of a target and a phenotype (e.g. expression of virulence factors). Examples of valid detection assays include, but are not limited to, detection assays that, when a target is detected, accurately predict the virulence phenotype 95%, 96%, 97%, 98%, 99%, 99.5%, 99.8%, or 99.9% of the time. Other examples of valid detection assays include, but are not limited to, detection assays that qualify as and/or are marketed as Analyte-Specific Reagents (i.e. as defined by FDA regulations) or In-Vitro Diagnostics (i.e. approved by the FDA).

As used herein, the term “kit” refers to any delivery system for delivering materials. In the context of reaction assays, such delivery systems include systems that allow for the storage, transport, or delivery of reaction reagents (e.g., microarrays, oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain a microarray for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to an organism” refers to facts or data pertaining to an organism (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc. “Allele frequency information” refers to facts or data pertaining allele frequencies, including, but not limited to, allele identities, statistical correlations between the presence of an allele and a characteristic of a subject (e.g., a human subject), the presence or absence of an allele in a individual or population, the percentage likelihood of an allele being present in an individual having one or more particular characteristics, etc.

As used herein, the term “assay validation information” refers to genomic information and/or allele frequency information resulting from processing of test result data (e.g. processing with the aid of a computer). Assay validation information may be used, for example, to identify a particular candidate detection assay as a valid detection assay.

### DETAILED DESCRIPTION OF THE INVENTION

The present invention relates to compositions and methods for the detection and characterization of nucleic acid sequences and variations in nucleic acid sequences present in multiple genomes. In particular, the present invention provides microarrays possessing two or more whole genomes and methods of making and using the same to detect the presence or absence of a target sequences in the plurality of genomes.

Identifying the functional and biological significance of genes and their alleles is fundamental to interpreting data derived from genomic studies. Comparing gene frequencies among isolates collected from different sources (e.g., disease causing and commensal isolates) serves as a valuable strategy to gain insight into the relative importance of a gene sequence in pathogenesis, transmission and other biologically significant properties (See e.g., Zhang et al., Infect Immun, 68, 2009, (2000)). Microarray technology has proven to be a powerful tool in this regard. Current DNA microarray platforms are used to gain insights into gene function and gene interactions using two experimental paradigms: 1) mRNA profiling to provide a global survey of gene activity; and 2) comparative genome scans for global surveys of genetic variants (See e.g., Harrington et al., Curr Opin Microbiol, 3, 285 (2000); Fitzgerald and Musser, Trends Microbiol, 9, 547 (2001); Schoolnik, Curr Opin. Microbiol, 5, 20 (2002)).

However, currently available arrays contain probe sequences representing all or most genes of a single, annotated genome. Hence, current genome scans are limited to the genetic features present in the genome of the arrayed reference strain. Given the substantial differences among the sequence repertoires of various strains of a single species (See e.g., Dougan et al., Curr Opin Microbiol, 4, 90 (2001)), a uniform, comprehensive genome scan for any given species (e.g., bacterial, viral, etc.) has not been forthcoming. For example, in order to scan the genomes of 5000 different isolates of a pathogen, at least 5000 different microarrays would need to be made and analyzed. Hence, the associated cost and complexity of data acquisition of the current microarray platforms and their methods of use limits current studies to a small number of samples.

Comparative genome scanning has provided limited insight into both the evolution of pathogens and the overall differences between pathogenic and commensal organisms of the same species (See e.g., Schoolnik, Curr Opin. Microbiol, 5, 20 (2002); Welch et al., Proc Natl Acad Sci USA, 99:17020 (2002); Whittam and Bumbaugh, Curr Opin Genet Dev, 12:719 (2002)). However, the study of large numbers of strains is required to determine the relative frequency of various genes within a species and to gain insight into their association with pathogenesis, antibiotic resistance, adaptation to environmental factors, and transmission. Large population based samples are required to minimize the identification of spurious associations that often arise with small and convenient sample comparisons.

The present invention provides assays that can be performed on large numbers of entire genomes, simultaneously, to detect for the presence or absence of gene content responsible for biological properties. Accordingly, in some embodiments, the present invention provides a composition comprising two or more genomes affixed to a solid surface. In other embodiments, the present invention provides a composition comprising a plurality of whole genomes provided as a microarray on a solid surface (e.g., see Example 2).

The present invention also provides an effective high throughput method for genome isolation from numerous samples for array printing (See, e.g., Example 4). In some embodiments, this method provides highly concentrated and fragmented genomic nucleic acid using sonication and heat treatment. In some embodiments, the genomic nucleic acid is DNA. In some embodiments, the genomic nucleic acid is RNA. In some embodiments, the genomic nucleic acid is both DNA and RNA. The present invention provides a new and robust bacterial genomic DNA isolation method with minimal cost. The method involves only a few steps and can be performed in a high throughput format. In some embodiments, the methods can be automated. Thus, in some embodiments, the method finds use for generating a plurality of genomes suitable for use in the methods and compositions of the present invention, as well as providing efficient methods of preparing DNA for conventional microarray comparative genomic experiments and routine PCR amplification.

The present invention provides multiple approaches to determine the presence or absence of nucleic acid sequence in a plurality of genomes. In some embodiments, the composition of two or more genomes affixed to a solid surface comprise total genomic nucleic acid. In other embodiments, the two or more genomes comprise total genomic DNA or total genomic RNA. In further embodiments, the total genomic DNA or total genomic RNA comprises DNA or RNA, derived from a single individual, strain, isolate, or species. In still further embodiments, the single individual, strain, isolate or species is selected from the group comprising humans, bacteria, viruses, yeast, algae, fungi, animals and plants.

When used directly for printing onto an array, purified total genomic nucleic acids (e.g., DNA) produce very weak hybridization signals due in part to inefficient binding of long DNA molecules to solid surfaces. The present invention provides methods for overcoming this limitation, permitting the arraying and use of multiple genomes on a single surface. In an effort to decrease the viscosity of the DNA solution and to improve the spread and binding of total genomic nucleic acid to a solid surface, additional purification and treatment steps can be carried out (See, e.g., Examples 1 and 4). Accordingly, in some embodiments, the total genomic DNA or total genomic RNA is highly purified. In some embodiments, the purification comprises organic extraction. In some embodiments, the purification comprises the use of membranes and resins. In a preferred embodiment, the two or more genomes are fragmented. In some embodiments, the fragmentation is performed using sonication (See, Example 4). In some embodiments, the fragmented genomes are substantially composed of fragments 0.1 kb-10 kb in length. In other embodiments, the fragments are 1.0 kb-10 kb in length. In still other embodiments, the fragments are 2.0 kb-10 kb in length. In a preferred embodiment, the fragments are 2.0 kb-5.0 kb in length.

Once each of the two or more genomes are fragmented, the genomes are affixed to a solid surface (e.g., see Example 1). In some embodiments, the solid surface to which the two or more genomes are affixed is glass. In some embodiments, the glass is a glass slide. Solid surfaces may be treated. The present invention is not limited to a particular method of fabricating or type of array. Any number of suitable chemistries known to one skilled in the art may be utilized (e.g., amine or epoxy modified surface arrays, see Example 1).

Furthermore, the present invention is not limited by the type of solid surface chosen. Indeed, a variety of solid surfaces find use in the present invention, including, but not limited to, silicon, plastic, polymer, ceramic, photoresist, nitrocellulose, hydrogel, paper, polypropylene, polystyrene, nylon, polyacrylamide, optical fiber, natural fibers, nylon, metals, rubber and composites thereof. In preferred embodiments, the solid surface is nylon (e.g., nylon polymers, See, e.g., Example 5). In some embodiments, the solid surfaces are patterned for attachment of biological macromolecules (e.g., nucleic acids). In some embodiments, the solid surface is planer. The present invention is not limited to a particular type of solid surface. In some embodiments, the solid surface further comprises a plurality of etched microchannels. In other embodiments, the solid surface is in a two-dimensional configuration or a three-dimensional configuration comprising pins, rods, fibers, tapes, threads, sheets, films, gels, membranes, beads, plates, particles, microtiter wells, capillaries, or cylinders.

The present invention is not limited to the array fabrication methods described above. Additional array generating technologies may be utilized, including, but not limited to, those described below.

An array of two or more genomes may be constructed by electronically capturing the genomes on the solid surface (Nanogen, San Diego, Calif.) (See e.g., U.S. Pat. Nos. 6,017,696; 6,068,818; and 6,051,380; each of which are herein incorporated by reference). Alternatively, a modified method of Nanogen's technology, which enables the active movement and concentration of charged molecules to and from designated test sites on a semiconductor microchip is utilized. Genomes are electronically placed at, or “addressed” to, specific sites on the solid surface. Since nucleic acids (e.g., DNA) has a strong negative charge, it can be electronically moved to an area of positive charge. In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Common reagents and washes are delivered by flooding the entire surface and removing by spinning. A plurality of genomes can be affixed to the solid support using Protogene's technology.

In some embodiments, the present invention provides a plurality of whole genomes provided as a microarray on a solid surface. In preferred embodiments, microarrays comprise at least 10, preferably at least 100, even more preferably at least 1,000, still more preferably, at least 3,000, even more preferably, least 10,000, and yet more preferably, at least 30,000 distinct genomes. In preferred embodiments, each distinct genome is affixed to a specific location on the microarray. In preferred embodiments, the solid surface size to which the plurality of genomes is affixed is 20 mm×60 mm or smaller.

In some embodiments, the present invention provides a nucleic acid array, the nucleic acid array comprising a solid support and a plurality of whole genomes, each of the whole genomes affixed to the solid support at a predetermined location, and each of the whole genomes comprising total genomic DNA or RNA, the total genomic nucleic acid (e.g., DNA) derived from a single individual, strain, isolate or species of humans, bacteria, viruses, yeast, algae, fungi, animals or plants, wherein the total genomic DNA or RNA is fragmented. The present invention provides the use of whole genomes comprising total genomic nucleic acid (e.g., DNA) from a variety of bacteria, including, but not limited to, Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasm, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes. Likewise, the present invention provides the use of whole genomes comprising total genomic nucleic acid (e.g., DNA) from a variety of viruses, including, but not limited to human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses. The present invention also provides the use of whole genomes comprising total genomic DNA or RNA from a variety of fungi, including, but not limited to Cryptococcus neaformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis.

As discussed above, in some embodiments, the present invention uses established cDNA glass microarray fabrication and hybridization techniques, but instead of homogenous DNA of single genes or single genomes, total genomic nucleic acid (e.g., DNA) of two or more genomes is printed on a solid surface. This approach results in the target sequence (that sequence within the plurality of genomes being interrogated by a probe) representing a tiny fraction of the total genome fragments in each spot. Thus, detection sensitivity is a major concern. Hybridization signal strength is determined by both the target concentration in the spot and the quantity of the label carried by the probe. In standard microarray assays, fluorescent dye is incorporated into the DNA probe by an enzymatic reaction. The longer the probe, the more dye molecules it will eventually carry.

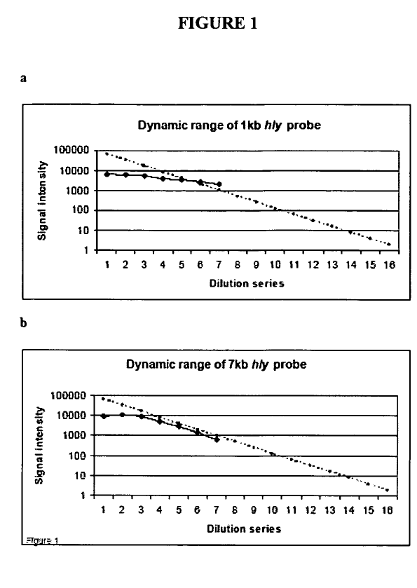

In order to determine hybridization sensitivity, an array with a two fold dilution series of a genomic DNA sample (prepared as describe in Example 1) was printed onto a glass slide and hybridized with either a 1 kb or 7 kb Cy5 directly-labeled DNA probe. Signals were detectable for the 1 kb Cy5 probe but without valid dynamic range (e.g., see Example 2, FIG. 1A). When the same array was hybridized with a 7 kb Cy5 labeled DNA probe, the hybridization signal was significantly increased due to a higher number of dye molecules incorporated into the hybridizing probe (e.g., see Example 2, FIG. 11B).

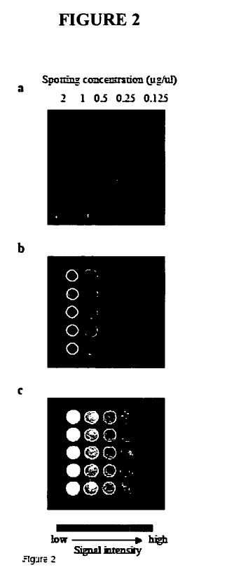

When using probes ranging in size from a few hundred base pairs to 2 kb, signal amplification is often necessary for detecting the target sequence in the plurality of genomes present on a solid surface. DNA dendrimer (3DNA reagent) and Tyramine Signal Amplification System (TSA) were used to increase detection sensitivity. A 3DNA dendrimer is a signal amplification molecule made from DNA. Each 3DNA molecule contains an average of 375 fluorescent dye molecules and can bind to any sized DNA probe with a capture sequence at its end. A 1 kb dendrimer probe generated a much higher signal than a 1 kb directly-labeled probe (e.g., see Example 2, FIG. 2B and A, respectively). ssDNA dendrimer probes were prepared using a ssDNA fragment generated by λ exonulcease treatment. The single stranded dendrimer probe eliminated probe self hybridization, enhancing probe and target hybridization kinetics, thereby generating stronger and more consistent hybridization signals. TSA is an enzyme-based secondary signal amplification system. The TSA system produced much stronger signals than the dendrimer probe (e.g., see Example 2, compare FIGS. 2C and 2B).

Accordingly, the present invention provides a method for detecting a target sequence in a plurality of genomes comprising providing a composition comprising two or more genomes affixed to a solid surface; a probe specific for a target sequence; and hybridizing the probe to the composition under conditions such that the presence or absence of the sequence in the two or more genomes is identified. In some embodiments, the target sequence in the plurality of genomes comprises nucleic acid sequence. In a preferred embodiment, the genomes comprise genomes from pathogens. In other preferred embodiments, the target sequence is a gene associated with antibiotic susceptibility or resistance. In some embodiments, the target sequence is a transposable element. In still other embodiments, the target sequence encodes all or part of a nucleic acid sequence of interest, including, but not limited to, sequences of virulence genes, antibiotic resistant genes, transposable elements, genes with single nucleotide mutations, genes with single nucleotide polymorphisms, genes with deletions, genes with insertions, and genes with mutations.

A number of methods are employed to overcome the detection sensitivity issue discussed above. In a preferred embodiment, the probe contains a dendrimer capture sequence. In other preferred embodiments, the probe is detectably labeled with fluorescent dyes. In a particularly preferred embodiment, the fluorescent dyes include, but are not limited to, fluorescein dyes, rhodamine dyes, BODIPY, and Cy3 or Cy5 dyes The present invention is not limited to a particular type of label. Indeed, a variety of detectable labels find use in the present invention including biotin, magnetic beads, radiolabels, enzymes, colorimetric labels and plastic beads.

In a preferred embodiment, the probe specific for a target sequence is single stranded DNA. The present invention is not limited by the nature of the probe used. Indeed a variety of probes find use in the present invention including an oligonucleotide, DNA, amplified DNA, cDNA, double stranded DNA, PNA, RNA, and mRNA. In some embodiments, the probe is less than 100 bp. In other embodiments, the probe is 0.1 kb-1.0 kb. In still other embodiments, the probe is 1.0 kb-5.0 kb. In other embodiments, the probe is 5.0 kb-7.0 kb. In some embodiments, the probe is 7.0 kb-10 kb. In some embodiments, the probe is greater than 10 kb.

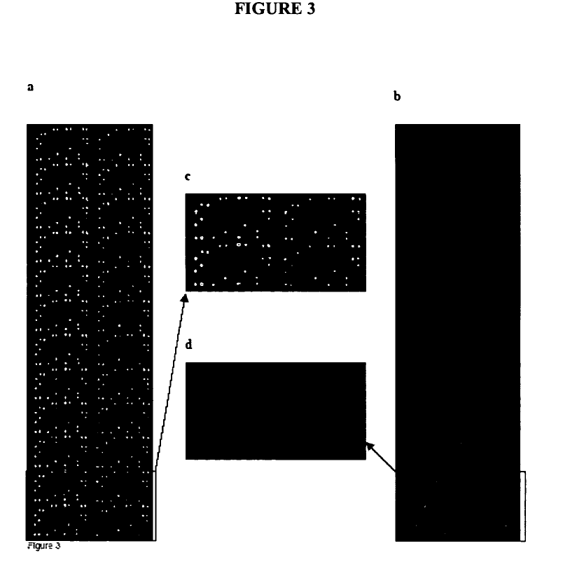

To detect the presence or absence of a target sequence in each genome spot on the array, in some embodiments, signals generated from target sequences within the plurality of genomes were compared to a positive control. Therefore, it was important that the same number of copies of each genome be compared. Although all genomic DNA samples were suspended in the spotting buffer at the same concentration before arraying, they still could differ in genome copy number per spot due to genome size and plasmid content variations. In addition, exact amounts of DNA fixed in each spot could vary due to technical limitations during the printing and post-print processes. In order to account for these variations, in some embodiments, the identification of the presence or absence of the target sequence in the plurality of genome is standardized using a dual channel non-competing hybridization strategy. In further embodiments, the dual channel non-competing hybridization strategy utilizes signals generated by 16s rRNA (e.g., see Example 3, FIGS. 3A and B).

In some embodiments, the present invention provides a method for comparing genomes for the presence or absence of one or more sequences, the method comprising contacting a microarray comprising a plurality of whole genomes derived from different sources with one or more nucleic acid probes and identifying the genome or genomes to which the probe(s) binds. It is contemplated that such a method will permit one to examine the extent of shared genetic elements across species, especially horizontally transferred virulence factors and antibiotic resistance genes Furthermore, such a method also permits the simultaneous analysis of two or more genomes for detecting sequences of virulence genes, antibiotic resistant genes, transposable elements, genes with single nucleotide mutations, genes with single nucleotide polymorphisms, genes with deletions, genes with insertions, and genes with mutations. In some embodiments, the microarray comprises two or more genomes derived from a single type of bacteria, virus, fungus, yeast or algae, but under different forms of environmental stress. In further embodiments, the environmental stress comprises heat shock, low temperature, amino acid depletion, ultraviolet radiation or exposure to antibiotics.

The invention also provides a kit comprising a composition comprising a plurality of whole genomes provided as a microarray on a solid surface. In some embodiments, the kit comprises instructions for using the microarray, wherein the instructions are for determining the presence or absence of a target sequence within one or more of the plurality of whole genomes. In other embodiments, the kit comprises probes specific for binding to a target sequence within one or more of the plurality of whole genomes. In further embodiments, the probe is selected from a group consisting of an oligonucleotide, DNA, amplified DNA, cDNA, single stranded DNA, double stranded DNA, PNA, RNA, and mRNA.

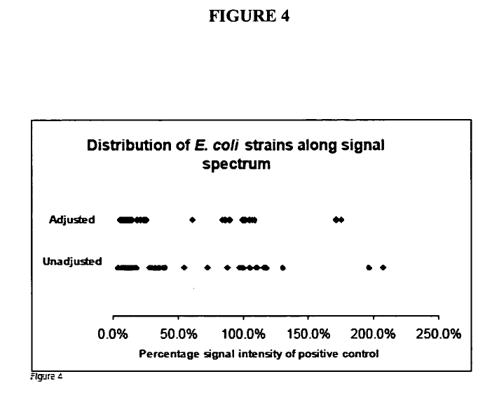

Low density (around 2,000 spots) and high density (around 15,000 spots) arrays were generated on a 22 mm×60 mm surface by replicate spotting of the E. coli ECOR collection (Ochman and Selander, J Bacteriol, 157, 690 (1984)) using the methods discussed above (e.g., see Example 3). The isolates were screened for the presence or absence of E. coli virulence genes. Data generated was compared to previous results obtained by other methods. The results of hemolysin gene (hly) hybridizations are shown (see Example 3, FIGS. 3-4).

Accordingly, the present invention also provides a method of making an array wherein two or more genomes are affixed to a solid surface.

Experimental

The following examples are provided in order to demonstrate and further illustrate certain preferred embodiments and aspects of the present invention and are not to be construed as limiting the scope thereof.

In the experimental disclosure that follows, the following abbreviations apply: g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); U (units), kb (kilobase); bp (base pair); hr (hour); min (minute); MoBio (Mo Bio Laboratories, Inc., Carlsbad, Calif.); Qiagen (Qiagen, Santa Clarita, Calif.); Promega (Promega Corporation, Madison, Wis.); Millipore (Millipore Inc., Billerica, Mass.); Misonix (Misonix Inc., Farmingdale, N.Y.); Bio-Rad (Bio-Rad Inc., Hercules, Calif.); TeleChem (TeleChem Inc., Sunnyvale, Calif.); Invitrogen (Invitrogen Corp., Carlsbad, Calif.); Novagen (Novagen, Madison, Wis.); Corning (Corning Inc., Acton, Mass.); Genisphere (Genisphere, Inc., Hatfield, Pa.); PerkinElmer, (PerkinElmer Inc., Boston, Mass.); Molecular Dynamics (Molecular Dynamics Inc, Sunnyvale, Calif.); Greiner Bio-one, (Greiner Bio-one, Longwood, Fla.); and TeleChem (TeleChem Inc., Sunnyvale, Calif.).

### EXAMPLE 1

### Materials and Methods

DNA isolation and arraying. Due to the heterogeneous nature of DNA fragments within a total bacterial genomic preparation, genomic DNA was purified. Various DNA purification methods were performed including organic extraction and non-organic extraction methods based on membranes or resins. High quality DNA was obtained from each method and was suitable for array printing. Bead beating based lysing followed by a commercial DNA purification column worked the best for both Gram negative and Gram positive bacteria. For experiments that had a limited number of strains involved, DNA was isolated using QIAGEN Genomic-tip 20/G (Qiagen), UltraClean microbial DNA kit (MoBio), and Wizard Genomic DNA purification kit (Promega) with an additional phenol extraction step. For DNA isolation from a large number of strains, the UltraClean-htp 96 well microbial DNA kit (MoBio) combined with MultiScreen Plate (Millipore) was used. This system combines bead beating lysis with a vacuum based membrane column. An additional step was used to remove precipitated debris and protein particles using the 96 well MultiScreen lysate clearing plate before loading the column. A MultiScreen PCR plate in a 96 well format was used to concentrate the eluted DNA. DNA concentration was determined by UV absorbance (260 nm) reading. For high throughput operation, genomic DNA was fragmented using sonication within wells of a 96 well microplate. DNA was fragmented using a Sonicator 3000 with a plate horn (Misonix) at amplitude setting of 10 for 8 min (rest 1 min for every 1 min on). For convenience, DNA samples were mixed with 2× commercial printing buffer prior to printing onto slides. A VersArray ChipWriter Compact system (Bio-Rad) was used to spot DNA onto SuperAmine glass slides (TeleChem) using either solid pin for low density printing or stealth pin, for high density printing. Using these methods, around 30,000 whole genome spots on a 20 mm×60 mm glass surface was the maximum density attainable with satisfactory hybridization results.

Probe labeling and array hybridization. Random priming was used to incorporate Cy3, Cy5, fluorescein, or biotin into dsDNA probes using the BioPrime DNA labeling system (Invitrogen) with appropriate dNTP mixtures. To prepare ssDNA used as dendrimer probe, DNA was first amplified by a pair of gene-specific primers. One primer had a manufacture specified capture sequence at the 5′ end and the other had a phosphorylated 5′ end. The dsDNA PCR product was then treated with λ exonuclease using a Strandase Kit (Novagen) to digest one strand of duplex DNA from the 5′ phosphorylated end to generate a ssDNA probe. All labeled probes were cleaned with a Qia-quick PCR purification kit (Qiagen). To prepare the hybridization mixture, 500 ng of each probe and 2 ug denatured salmon sperm DNA were mixed with 1.25× HybIt buffer (Telechem) to a final volume of 50 ul for each slide. The probes were denatured at 95° C. for 3 min and pipetted onto arrays, cover slips were applied, and the slides were placed in a hybridization chamber (Corning). Arrays were incubated at 63° C. in a water bath for 18-24 hr and then washed according manufacture's directions. A 3DNA Submicro Expression Array Detection Kit (Genisphere) was used for subsequent dendrimer hybridization and a MICROMAX TSA labeling and detection kit (PerkinElmer) was used for TSA signal amplification. In both cases, manufacture's protocols were followed. Detailed information of these two labeling and detection systems can be found at: http://www.genisphere.com/array_detection_faqs.html and http://las.perkinelmer.com/catalog/Category.aspx?CategoryName=MICROMAX, respectively, and are incorporated herein by reference.

Array scanning and data acquisition. Arrays were scanned with a VersArray ChipReader (Bio-Rad) at 10 μm resolution and variable photomulipier tube (PMT) voltage settings to obtain the maximal signal intensities with no saturation. When comparing signals of different hybridization conditions, the PMT and sensitivity settings of the scanner were kept at the same level. The resulting images were analyzed using either accompanied VersArray Analyzer software or ImageQuant Version 5.2 (Molecular Dynamics). To determine the presence or absence of hly gene (Cy3 signal) on the ECOR array (e.g., see Example 3), the percentage signal intensity relative to the positive control of each strain was calculated with and without DNA concentration adjustment based on 16s rRNA gene hybridization signal (Cy5 signal). The unadjusted percentage was calculated as Cy3 signal of the sample dividing by the average Cy3 signal of the positive controls. The adjusted percentage was calculated as Cy3/Cy5 signal ratio of the sample multiplied by the average Cy5 signal of the positive control, divided by the average Cy3 signal of the positive control. Based on an early study examining the sensitivity and specificity of different classification criteria (Zhang et al., J Microbiol Method, 44, 225(2001)), 50% was used as a cutoff point for differentiating hly positive and negative strains as it was the optimal breakpoint for classifying for the presence or absence of hly gene.

### EXAMPLE 2

### Array Hybridization and Detection

A test array with a two fold dilution series of a genomic DNA sample was printed and hybridized with either a 1 kb or a 7 kb Cy5 directly-labeled DNA probe (FIG. 1, A and B, respectively). No hybridization signal gain was observed beyond 1 ug/ul to 2 ug/ul of spotting concentration, indicating saturation of the binding capacity of the glass slide above this concentration. DNA concentrations above this limit resulted in decreased signal possibly due to washing off of DNA that was not directly bound during the hybridization process. Given the limited capacity of the glass surface for immobilizing DNA, the 1 kb Cy5 labeled probe generated very weak signals under standard instrument settings. By increasing the laser power and detector sensitivity, measurable signals were obtained, but without a valid dynamic range (FIG. 1A). When the same array was hybridized with a 7 kb Cy5 labeled DNA probe, the hybridization signal was significantly increased, and a linear response of the signal intensity along the concentration gradient was observed in the low concentration range (FIG. 1B).

When using probes ranging in size from a few hundred base pairs to 2 kb, signal amplification was necessary for detecting the target sequence on the array. DNA dendrimers (3DNA reagent) and the Tyramine Signal Amplification System (PerkinElmer) were used to increase detection sensitivity. A 1 kb dendrimer probe generated a much higher signal than a 1 kb directly-labeled probe (FIG. 2B and A, respectively). Initially, the dendrimer probe was prepared using dsDNA fragment. However, consistently strong signals were not obtained with the dsDNA dendrimer probe. Therefore, ssDNA dendrimer probes were prepared using a ssDNA fragment generated by λ exonulcease treatment. The single stranded dendrimer probe eliminated probe self hybridization, enhancing probe and target hybridization kinetics, thereby generating stronger and more consistent hybridization signals. For the TSA system, the probe was first labeled with either fluorescein or biotin, hybridized with the array, and then detected using antibody-horseradish peroxidase conjugate that catalyzed the deposition of Cy3 or Cy5 labeled tyramide reagent. The TSA system produced much stronger signals than the dendrimer probe (FIGS. 2C and B, respectively). The TSA system generated the most consistent and robust signals for detecting hybridization despite an elevated background and the need for extra incubation and washing steps.

### EXAMPLE 3

### E. coli Test Library Array

In order to test the optimized methods discussed in Examples 1 and 2 above, an array was created using the E. coli ECOR collection (Ochman and Selander, J Bacteriol, 157, 690 (1984)). Low density (around 2,000 spots) and high density (around 15,000 spots) arrays were generated on a 22 mm×60 mm surface by replicate spotting of these strains. The isolates were screened for the presence or absence of E. coli virulence genes. Data generated was compared to previous results obtained by other methods. The results of hemolysin gene (hly) hybridizations are shown (FIGS. 3-4).

In order to standardize signal intensity, DNA quantity present in each printed spot was observed employing a dual channel non-competing hybridization strategy using multiplex labeling and a multichannel laser scanner. One channel detected signal from the quantification probe, a Cy5 dye-labeled probe for the 16s ribosomal RNA gene present in all strains of the E. coli species in the same copy number, and a second channel detected signal from a probe for a target sequence, a Cy3 dye-labeled hly probe for this example. Since the genome quantification probe and the target sequence probe recognize different sequences within the genome, they are used in the same hybridization process. Hybridization results are obtained by scanning the slide at a different wavelengths, since Cy3 and Cy5 dyes are non-interfering dyes that excite at different wave lengths (FIGS. 3A and B). The 16s rRNA gene probe recognizes the same number of target sequences per genome of every sample. Therefore, its hybridization signal intensity was considered an indicator of genome quantity and used for hly hybridization signal adjustment using the Cy3/Cy5 signal ratio. Signal intensity of the quantification probe was normalized to the positive control, a ratio determined, and used to determine the presence or absence of the target sequence of interest, defined on the basis of a cutoff point established in a previous study (Zhang et al., J Microbiol Method, 44, 225(2001)). Using a 50% cutoff point, twelve strains were identified as hly gene positive, 100% congruent with results based on dot blot and Southern hybridization.

When plotted, the normalized signal intensities relative to the positive controls of these strains produced two more narrowly defined clusters around positive and negative control strains than did the unadjusted intensities (FIG. 4). Hence, the normalization process lead to more robust classification as these two clusters were more separated.

### EXAMPLE 4

### Rapid Bacterial Genomic DNA Isolation

Isolation of high quality genomic DNA is an important step in the bacterial comparative genomic studies using microarray of the present invention. Before the present invention, this step has usually been accomplished by employing in-house protocols or commercial kits or the like. Briefly, these processes involves multiple, time consuming steps, often including the handling of hazardous chemicals (See, e.g., Ausubel et al., Current Protocols in Molecular Biology. John Wiley and Sons. NY, (1994); Sambrook, et al., Molecular Cloning: A Laboratory Manual, 2^{nd} Ed. CSH Laboratory Press, Cold Spring Harbor, N.Y. (1989)). Further, the DNA preparation remains a manageable task since, in most cases, genomic nucleic acid is prepared from only a very limited number of strains, and only small fraction of the total genome of any given sample is used in any given array.

As the present invention provides compositions and methods that comprise libraries of entire genomes (e.g., 100, 1000, or 10,000 genomes) on a solid surface, the present invention also provides an effective high throughput method for genome isolation from numerous samples for array printing. In some embodiments, this method provides highly concentrated and fragmented bacterial DNA using sonication and heat treatment.

This new method was applied on both gram negative (Escherichia coli, Haemophilus influenzae) and gram positive (Streptococcus agalactiae) bacteria. Bacterial strains were grown overnight in 3 ml of liquid medium of choice in 10 ml culture tubes for small batch processing or in 96 deep-well plates (two plates with 1.5 ml per well inoculants were later combined) for high throughput processing. Bacteria were pelleted by centrifugation (20 min at 2000×g) and resuspended in 80 μl sonication buffer (50 mM Tris and 10 mM EDTA, pH 7.5; with optional 100 ng/μl RNase A). Resuspension was transfer to a 0.5 ml thin wall PCR tube or a fully skirted 96 well PCR plate (Greiner Bio-one). Tube/plate was placed in a plate horn (Misonix), filled with a water and ice mixture and treated with sonication using the Sonicator 3000 (Misonix) connected to the horn. Six treatments of 1 min each at amplitude setting of 6 for E. coli and H. influenzae and 10 for S. agalactiae were performed. The disrupted cell was then brought down to the bottom of the tube/plate by centrifugation (20 min at 2500×g). The tube/plate was then incubated in 98° C. water bath or a thermocycler for five minutes to precipitate out proteins in the supernatant by heat denaturing. After centrifugation (20 min at 2500×g), about 50 μl clean genomic DNA (and genomic RNA if RNase A is not added), already broken down to small fragments, was transferred to a new tube/plate and ready to be used for array printing. In some embodiments, a step for further purification and concentration was performed using a Microcon YM30 or a 96 well MultiScreen-PCR plate (Millipore, Mass.) to eliminate degraded RNA and to re-suspend the DNA in a new low salt buffer or water.

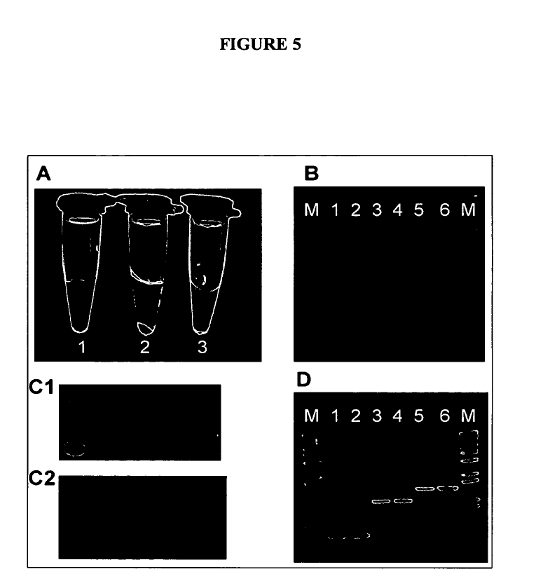

By applying sonic energy outside the sample tube/plate, direct contact of metal sonication probe with bacterial cells was avoided, thus eliminating potential contamination and made the high throughput sample processing in 96 well plates possible. This sonication treatment disrupted cell surface structures to release genomic DNA and RNA and yet did not disintegrate bacteria cells into clear lysate (See FIG. 5A, Tube 1). Therefore, most cell debris can be eliminated by centrifugation leaving relative clean supernatant with primarily nucleic acid and soluble components such as proteins. The soluble impurity can be further precipitated out by heat treatment (See, e.g., FIG. 5A, Tubes 2 and 3) leaving behind even purer DNA as reflected in the UV absorbance readings (See, Table 1).

TABLE 1
Concentrations and UV absorbance readings of DNA samples before and after heat treatment. Each
sample has three replicates and mean and ± standard deviation (SD) are show here.

| | Before heat treatment | | | After heat treatment | | |
|---|---|---|---|---|---|---|
| | Concentration | | | Concentration | | |
| Sample | (μg) | A₂₆₀/A₂₃₀ | A₂₆₀/A₂₈₀ | (μg) | A₂₆₀/A₂₃₀ | A₂₆₀/A₂₈₀ |
| 1 | 2.08 ± 0.15 | 1.32 ± 0.03 | 1.67 ± 0.02 | 1.72 ± 0.10 | 2.02 ± 0.05 | 1.86 ± 0.05 |
| 2 | 2.31 ± 0.12 | 1.33 ± 0.04 | 1.58 ± 0.04 | 2.01 ± 0.13 | 2.03 ± 0.04 | 1.92 ± 0.03 |
| 3 | 3.30 ± 0.20 | 1.49 ± 0.05 | 1.61 ± 0.06 | 2.54 ± 0.12 | 1.96 ± 0.10 | 1.88 ± 0.08 |

While absorbance reading is not a definitive assessment, it gives an indication of quality and purity (See, e.g., Sambrook, et al., Molecular Cloning: A Laboratory Manual, 2^{nd} Ed. CSH Laboratory Press, Cold Spring Harbor, N.Y. (1989)). Both A_{260/230} and A_{260/280} ratios increased after heat treatment indicating decreased impurities such as proteins and salts precipitation. Very high yield of DNA was obtained at the end and DNA samples all had uniformed sizes mostly between 100 bp to 1 kb (FIG. 5B). In some embodiments, the length of the nucleic acid can be increased or decreased based on the amplitude setting and treatment exposure time of the samples to the plate horn. For example, in some embodiments, the length of the nucleic acid (e.g., DNA) is 100 bp-1 kb. In other embodiments, the length of the nucleic acid (e.g. DNA) is 1 kb-2.5 kb. In still other embodiments, the length of the nucleic acid (e.g., DNA) is 1 kb-10 kb. Thus, in preferred embodiments, the resulting DNA does not require an additional fragmentation step before used for microarray experiments.

### EXAMPLE 5

### Test Library Array

To test the purified genomic DNA prepared in Example 4, DNA samples were mixed with DMSO (1:1) and printed onto a SuperAmine slide (TeleChem) using a VersArray ChipWriter system (Bio-Rad). Using the methods described in Examples 1-3, the resulting array was hybridized with a labeled DNA probe resulting in the attainment of a high quality hybridization result (See, FIG. 5C, panel 1). When other printing buffers or epoxy coated slides were used, the optional column purification step can be used to eliminate Tris in the samples. For more conventional comparative genomic hybridization where the genomic DNA is to be labeled and hybridized to a gene array, an isolated E. coli genomic DNA (after Microcon YM30 purification) was labeled with Cy3 by random primer extension and hybridized with a test slide printed with a set of 8 PCR amplified ORFs where 7 of the 8 ORFs are present in this strain. The expected hybridization result was obtained from each spot (See FIG. 5C, panel 2).

Isolating a specific sequence from a bacterial genome by PCR is one of the most routine laboratory procedures. DNA prepared with this new method can also be used as a template for such application. For example, in some embodiments, by using 1 μl of 1:50 diluted DNA samples (without optional column purification) in a 100 μl standard PCR reaction, it is possible to successfully and consistently amplify DNA fragments of various sizes up to 1.5 kb (FIG. 5D). While the majority of the DNA fragments are less than 1 kb after sonication (using the settings and sample treatment times provided in Example 4), it seems that enough large DNA fragments are still left to serve as templates for PCR amplification of fragments larger than 1 kb. Thus, in some embodiments, this method produces genomic DNA suitable for DNA amplification.

Thus, the present invention provides a new and robust bacterial genomic DNA isolation method with minimal cost. The method involves only a few steps and can be performed in a high throughput format. In some embodiments, the methods can be automated. The method finds use for generating a plurality of genomes suitable for use in the methods and compositions of the present invention, as well as providing an efficient method of preparing DNA for conventional microarray comparative genomic experiments and routine PCR amplification.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A composition comprising two or more genomes affixed to a solid surface.

2. The composition of claim 1, wherein said two or more genomes comprise total genomic nucleic acid.

3. The composition of claim 1, wherein said two or more genomes comprise total genomic DNA or total genomic RNA.

4. The composition of claim 1, wherein said genomes are derived from two or more organisms.

5. The composition of claim 1, wherein said two or more genomes are fragmented.

6. The composition of claim 5, wherein said fragmented genomes are substantially composed of fragments 0.1 kb-10 kb in length.

7. The composition of claim 1, wherein said two or more genomes are spotted in arrays on said solid surface.

8. The composition of claim 7, wherein said solid surface size is 20 mm×60 mm or smaller.

9. The composition of claim 1, wherein at least 10 genomes are spotted in arrays on said solid surface.

10. A method for detecting a target sequence in a genome, comprising:
a. providing:
i. a composition comprising a plurality of whole genomes provided as a microarray on a solid surface; and
ii. a probe specific for a target sequence;
b. hybridizing said probe to said composition under conditions such that the presence or absence of said target sequence in said genome is identified.

11. The method of claim 10, wherein said genomes comprise genomes from pathogens.

12. The method of claim 10, wherein said target sequence is a gene associated with antibiotic susceptibility or resistance.

13. The method of claim 10, wherein said target sequence is a transposable element.

14. The method of claim 10, wherein said target sequence comprises all or part of a nucleic acid sequence of a virulence gene, an antibiotic resistant gene, a transposable element, a gene with a single nucleotide mutation, a gene with a single nucleotide polymorphism, a gene with a deletion, a gene with an insertion, and a gene with one ore more mutations.

15. The method of claim 10, wherein said probe is 1.0 kb-10.0 kb.

16. A method for isolating genomes from a plurality of samples, comprising:
a) providing said samples;
b) applying sonic energy to said samples without direct contact between the sonication device and said samples;
c) heating said samples for a set period of time;
d) applying centrifugation to said samples.

17. The method of claim 16, wherein said genomes are derived from two or more organisms.

18. The method of claim 16, wherein said two or more genomes are fragmented.

19. The method of claim 16, further comprising purifying and/or concentrating said genome.

20. The method of claim 16, wherein said heating comprises heating said samples to between 95-100° C. for between 2-10 minutes.
